(19) United States
(12) Patent Application Publication
(10) Pub. No.: US 2025/0290008 A1
(43) Pub. Date: Sep. 18, 2025

(54) PROCESS FOR SEPARATING POLYENES FROM MONOENES

(71) Applicant: INNOLIPID, AS, Ålesund (NO)

(72) Inventor: Asgeir SAEBØ, Ålesund (NO)

(21) Appl. No.: 18/862,221

(22) PCT Filed: May 3, 2023
(86) PCT No.: PCT/IB2023/000237

§ 371 Date: Nov. 1, 2024

Related U.S. Application Data
(60) Provisional application No. 63/337,686, filed on May 3, 2022.

(51) Int. Cl. C11B 7/00; A23D 9/013; A23D 9/04

(52) U.S. Cl. CPC C11B 7/0075; A23D 9/013; A23D 9/04

(57) ABSTRACT

The present invention provides methods for separating polyenes from monoenes as well as products produced by the methods, and in particular to methods that comprise cryo-injection of an alcohol into a mixed-lipid composition.

### CROSS-REFERENCE TO RELATED APPLICATION

This application claims the benefit of U.S. Prov. Appl. 63/337,686 filed May 3, 2022, the contents of which are incorporated by reference in its entirety.

### FIELD OF THE INVENTION

The present invention provides methods for separating polyenes from monoenes as well as products produced by the methods, and in particular to methods that comprise cryo-injection of a mixed-lipid composition into an alcohol.

### BACKGROUND OF THE INVENTION

Isolation of polyunsaturated fatty acid ethyl esters on one side from saturated and monoenoic fatty acid ethyl esters on the other side is challenging. Urea complexation is a well-known technology used in the industry when saturated and monoenoic acids comprise only a small fraction. This process comprises dissolving urea in warm methanol or ethanol along with fatty acid esters and subsequently cooling down the transparent homogenous mixture slowly. Saturated esters especially, but also monounsaturated esters will then form a complex with urea and the precipitated material is easily removed in a centrifuge. The solvent and excess urea will then have to be removed from the product (filtrate). If also precipitated material constitute a potential value, urea must be removed from this fraction as well. Use of urea as an aid is undesirable and alternative technologies are needed.

For some applications, chromatography, for instance fluid bed chromatography is feasible or at least the best-known option currently, but the method is rather expensive if large volumes are to be processed.

Precipitation of saturated material either as a dry fractionation method or as a method including use of a solvent is well known. Separation of fatty acids by this method has been proposed decades ago and the recommendations are to use free acids instead of ethyl esters due to the great advantage of the higher melting points of the free fatty acids. However, working with the ester form do have some preferential feasibilities, especially the convenient access to various industrial intermediates and also the increased the oxidative stability.

Low temperature crystallization in industrial scale do have obstacles not recognized in bench scale chemistry, and therefore despite numerous examples of cooling fatty acids and fatty acid ethyl esters in solvents has not materialized into feasible full-scale production. The only exception may be the method of removing the saturated fatty acid Palmitic acid from an ethyl ester blend described in U.S. Pat. No. 10,190,075 (1) In this patent a process is described wherein a small amount of ethanol (less than the weight of the esters) is added to an ethyl ester blend comprising mainly palmitic acid and palmitoleic acid and cooled and mixed thoroughly prior to removal of crystals in a centrifuge. This method was reported to be able to totally remove palmitic acid from the blend. However, despite focusing on enrichment of palmitoleic acid, the patent does not offer any solution for the removal of other fatty acid ethyl esters, typically comprising at least three different poly-unsaturated carbon 16 fatty acids. Hence, products in the market remains rather low in purity, and the commercially marketed products claiming to be the purest forms available comprise about 50% palmitoleic acid despite less than 1% palmitic acid. The saturates have apparently been removed successfully, but three major polyunsaturated C 16 fatty acid esters along with traces of other remain in the product and accounts for almost 50% of the product.

A recent paper in Journal of Oleo Science (2) teaches how a process combining molecular distillation and solvent crystallization yielded a palmitoleic acid product with a purity of 54%. Again, palmitic acid was easily removed, but no solution was offered for further enrichment of palmitoleic acid.

This invention addresses in general the removal of polyunsaturated fatty acids from monounsaturated fatty acids of equal chain length and in particular the removal of poly unsaturated fatty acids with 16 carbons from palmitoleic acid.

### SUMMARY OF THE INVENTION

The present invention provides methods for separating polyenes from monoenes as well as products produced by the methods, and in particular to methods that comprise cryo-injection of a mixed-lipid composition into an alcohol.

In some preferred embodiments, the present invention provides methods of isolating a fraction containing a monounsaturated fatty acid ethyl ester from a fraction containing polyunsaturated fatty acid ethyl esters of equal carbon-number comprising 1) cooling a mixture of said blend in ethanol to a temperature at which the monounsaturated fatty acid precipitates and 2) separating the precipitated fraction from the non-separated fraction and 3) removal of the ethanol solvent by vacuum distillation.

In some preferred embodiments, the present invention provides methods of separating a fraction containing a monounsaturated fatty acid ethyl ester from a fraction containing polyunsaturated fatty acid ethyl esters of equal carbon-number in a fatty acid ethyl ester blend containing the monounsaturated and polyunsaturated fatty acid ethyl esters of equal carbon-number comprising 1) cooling a mixture of the blend in ethanol to a temperature at which the monounsaturated fatty acid precipitates to provide a precipitated fraction comprising the monounsaturated fatty acid and a fraction comprising the polyunsaturated fatty acid ethyl esters, 2) separating the precipitated fraction from the non-separated fraction, and 3) removing the ethanol solvent by vacuum distillation to provide the fraction comprising polyunsaturated fatty acid ethyl esters.

In some preferred embodiments, the fatty acid ethyl ester blend and ethanol are combined in a ratio of between 1:3 (w/w) and 1:20 (w/w).

In some preferred embodiments, the fatty acid ethyl ester blend comprises fatty acid moieties having a length of 16 carbons and the fraction comprising polyunsaturated fatty acid ethyl esters comprises a blend of C16:2, C16:3 and/or C16:4 ethyl esters. In some preferred embodiments, the precipitated fatty acid ethyl ester is a C16:1 ethyl ester.

In some preferred embodiments, the fatty acid ethyl ester blend comprises fatty acid moieties having a length of 20 carbons and the fraction comprising polyunsaturated fatty acid ethyl esters comprises a blend of C20:2, C20:3, C20:4 and/or C20:5 ethyl esters. In some preferred embodiments, the precipitated fatty acid ethyl ester is a C20:1 ethyl ester.

In some preferred embodiments, the fatty acid ethyl ester blend comprises fatty acid moieties having a length of 22 carbons and the fraction comprising polyunsaturated fatty acid ethyl esters comprises a blend of C22:2, C22:3, C22:4 and/or C22:5 ethyl esters. In some preferred embodiments, the precipitated fatty acid ethyl ester is a C22:1 ethyl ester.

In some preferred embodiments, the fatty acid ethyl ester blend containing the monounsaturated and polyunsaturated fatty acid ethyl esters has been treated by a previous step where unsaturated fatty acid ethyl esters are removed or reduced in concentration. Thus, in some preferred embodiments, the fatty acid ethyl ester blend may be further characterized as comprising less than 20.0%, 10.0%, 5.0%, 4.0%, 3.0%, 2.0% or 1.0% w/w (or any range or value therein) of the unsaturated fatty acid ethyl ester species (e.g., C16:0, C18:0, C20:0, C22:0, C24:0 or C26:0 fatty acid ethyl ester).

In some preferred embodiments, the methods further comprise injecting the fatty acid ester blend into the ethanol. In some preferred embodiments, the injection of the fatty acid ethyl ester into the ethanol is performed through a nozzle or microtubular device.

In some preferred embodiments, the ethanol is cooled prior to injection of the fatty acid ethyl ester blend. In some preferred embodiments, the precooled ethanol is cooled to between minus 50° C. and minus 90° C. before injecting the fatty acid ethyl ester blend.

In some preferred embodiments, the fatty acid ethyl ester blend is sprayed through a nozzle that is cooled by simultaneously flushing fluid nitrogen through the nozzle.

In some preferred embodiments, the fatty acid ethyl ester blend that is injected into the cooled ethanol solution comprises a premixture of fatty acid ethyl esters and ethanol in a ratio of about 1:1 (w/w).

In some preferred embodiments, the fatty acid ethyl esters are derived from a vegetable oil or a marine oil. In some preferred embodiments, the marine oil is derived from a fish, algae, or krill.

In some further preferred embodiments, the present invention provides methods of fractionating lipids comprising a) dissolving the lipids in a solvent and b) spraying the dissolved lipids through a nozzle system to atomize the dissolved lipids and c) simultaneously flushing a stream of supercooled fluid air through the nozzle system to instantly cool the atomized blend and d) separating solid (precipitated) lipids from non-precipitated lipids. In some preferred embodiments, the air comprises more than 95% nitrogen. In some preferred embodiments, the solvent is selected from or comprise any blend of water, methanol, ethanol, propanol, butanol, hexane, iso-octane, heptane, and acetone. In some preferred embodiments, the lipids are derived from a vegetable oil or a marine oil. In some preferred embodiments, the marine oil is derived from a fish, algae, or krill.

In some preferred embodiments, the present invention provides a food supplement product comprising a product made by a method as described above.

In some preferred embodiments, the present invention provides a pharmaceutical product comprising a product made by a method as described above.

### DETAILED DESCRIPTION OF THE INVENTION

There are at least two major challenges working with low temperature crystallization in large scale. There is normally a need for a certain time to allow for crystals to form. Secondly, when cooling down huge volumes, lipids in the initial stage of precipitation tend to accumulate on the cooling surface which necessitates removal to allow for efficient cooling. For example, in a large-scale reactor, lipids must be scraped off the surface. This is an undesirable use of energy which also adds heat to the operation. Costs of cooling down to very low temperatures could potentially be reduced by integrating heat exchangers in the plant. A finished filtrated very cool blend could, for example, be warmed by the next batch to be cooled down by exchange of heat. A heat exchanger system can however not be applied if lipids precipitate on the surface in the heat exchanger.

The present invention provides methods that address these problems, and in methods that comprise cryo-injection of a mixed-lipid composition into an alcohol. Indeed, the present invention challenges the conventional perception of the necessity of allowing time for crystals to grow. One preferred embodiment of this invention provides a solution to precipitated lipids accumulating on surfaces of plate exchangers or on cooled reactor surface. Instead of cooling down a mixture of a lipid in the solvent, the solvent alone is cooled down, well below the temperature at which the crystals are desired to be harvested. Then the lipid blend, typically having a temperature far above the crystalized target temperature is injected as microdroplets into the supercooled solvent. The process is designed such that when the entire volume of lipid is injected, the temperature increase in the mixture will reach the predesigned “crystallization” temperature. In another embodiment of the invention, the lipid blend to be injected in the solvent may contain a small amount of said solvent to be able to reduce the temperature slightly before the blend is injected in the very low temperature ethanol solvent.

Injecting a room temperature lipid mixture into a very low temperature solvent will instantly produce crystals that also will trap some lipid droplets normally soluble in the solvent at the very low temperature. However, due to their small size, the soluble lipids will very rapidly separate from the crystalized lipids rendering the mixture suitable for filtration after a short time.

Further, in another embodiment of this invention, a pure palmitoleic acid (90% or greater w/w) is easily produced at a temperature of about −70° C. At this temperature, palmitoleic acid will precipitate whereas polyunsaturated carbon 16 fatty acids still will be soluble.

Accordingly, in some preferred embodiments, the present invention provides methods of isolating a fraction containing a monounsaturated fatty acid ethyl ester from a fraction containing polyunsaturated fatty acid ethyl esters of equal carbon-number in a fatty acid ethyl ester blend containing the monounsaturated and polyunsaturated fatty acid ethyl esters of equal carbon-number comprising 1) cooling a mixture of said blend in ethanol to a temperature at which the monounsaturated fatty acid precipitates to provide a precipitated fraction comprising the monounsaturated fatty acid and a non-separated fraction comprising the polyunsaturated fatty acid ethyl esters, 2) separating the precipitated fraction from the non-separated fraction, and 3) removal of the ethanol solvent by vacuum distillation.

In some preferred embodiments, the fatty acid ethyl ester blend and ethanol are combined in a ratio of between 1:3 and 1:20 w/w (fatty acid ethyl ester blend to ethanol on a weight/weight basis). In some preferred embodiments, the fatty acid ethyl ester blend and ethanol are combined in a ratio of between 1:5 and 1:15 w/w.

In some preferred embodiments, the fatty acid ethyl ester blend is introduced into the ethanol. The present invention is not limited to any particular method of introduced the fatty acid ethyl ester blend into the ethanol. In some preferred embodiments, the fatty acid ethyl ester blend is injected into the ethanol. In some particularly preferred embodiments, the injection of the fatty acid ethyl ester blend into the ethanol is performed through a nozzle or microtubular device. In some preferred embodiments, the ethanol is cooled prior to injection of the fatty acid ethyl ester blend. In some preferred embodiments, the precooled ethanol is cooled to between minus 50° C. and minus 90° C. before injecting the fatty acid ethyl ester blend. In some preferred embodiments, the fatty acid ethyl ester blend to be injected into the cooled ethanol solution comprises a premixture of the fatty acid ethyl ester blend and ethanol in a ratio of about 2:1 to 1:2 (w/w), more preferably in aa ratio of about 1:1, where about is +/−5% w/w, and most preferably at a ratio of 1:1 (w/w). In some still further preferred embodiments, the fatty acid ethyl ester blend is sprayed through a nozzle that is cooled by simultaneously flushing also fluid nitrogen through the nozzle.

The methods of the present invention are not limited to use with fatty acid ethyl ester blends containing fatty acid moieties of any particular equal lengths. In some embodiments, the fatty acid ethyl ester blends comprise, consist essentially of, or consist of monounsaturated and polyunsaturated fatty acid moieties of 16 carbons in length (e.g., C16:1, C16:2, C16:3 and/or C16:4). In these embodiments, the precipitated monounsaturated ethyl ester is a C16:1 ethyl ester and/or the non-separated fraction comprises polyunsaturated fatty acid ethyl esters (e.g., C16:2, C16:3 and/or C16:4 ethyl esters). In some embodiments, the fatty acid ethyl ester blends comprise, consist essentially of, or consist of monounsaturated and polyunsaturated fatty acid moieties of 18 carbons in length (e.g., C18:1, C18:2, C18:3 and/or C18:4). In these embodiments, the precipitated monounsaturated ethyl ester is a C18:1 ethyl ester and/or the non-separated fraction comprises polyunsaturated fatty acid ethyl esters (e.g., C18:2, C18:3 and/or C18:4 ethyl esters). In some embodiments, the fatty acid ethyl ester blends comprise, consist essentially of, or consist of monounsaturated and polyunsaturated fatty acid moieties of 20 carbons in length (e.g., C20:1, C20:2, C20:3, C20:4 and/or C20:5). In these embodiments, the precipitated monounsaturated ethyl ester is a C20:1 ethyl ester and/or the non-separated fraction comprises polyunsaturated fatty acid ethyl esters (e.g., C20:2, C20:3, C20:4 and/or C20:5 ethyl esters). In some embodiments, the fatty acid ethyl ester blends comprise, consist essentially of, or consist of monounsaturated and polyunsaturated fatty acid moieties of 22 carbons in length (e.g., C22:1, C22:2, C22:3, C22:4 and/or C22:5). In these embodiments, the precipitated monounsaturated ethyl ester is a C22:1 ethyl ester and/or the non-separated fraction comprises polyunsaturated fatty acid ethyl esters (e.g., C22:2, C22:3, C22:4 and/or C22:5 ethyl esters). In some embodiments, the fatty acid ethyl ester blends comprise, consist essentially of, or consist of monounsaturated and polyunsaturated fatty acid moieties of 24 carbons in length (e.g., C24:1, C24:2, C24:3, C24:4 and/or C24:5). In these embodiments, the precipitated monounsaturated ethyl ester is a C24:1 ethyl ester and/or the non-separated fraction comprises polyunsaturated fatty acid ethyl esters (e.g., C24:2, C24:3, C24:4 and/or C24:5 ethyl esters). In some embodiments, the fatty acid ethyl ester blends comprise monounsaturated and polyunsaturated fatty acid moieties of 26 carbons in length. (e.g., C26:1, C26:2, C26:3, C26:4, C26:5 and/or C26:6). In these embodiments, the precipitated monounsaturated ethyl ester is a C26:1 ethyl ester and/or the non-separated fraction comprises polyunsaturated fatty acid ethyl esters (e.g., C26:2, C26:3, C26:4, C26:5 and/or C26:6 ethyl esters).

In some embodiments, the fatty acid ethyl ester blends comprise monounsaturated and polyunsaturated fatty acid moieties of equal lengths of from 16 to 26 carbons. In some embodiments, the fatty acids ethyl ester blends comprise monounsaturated and polyunsaturated fatty acid moieties of equal length of from 16 to 22 carbons in length.

In some preferred embodiments, the processes described herein are the second step of a process where ethyl esters comprising an unsaturated fatty acid moiety (e.g., C16:0, C18:0, C20:0, C22:0, C24:0 or C26:0) are removed from the ethyl ester blend in a previous step, such as a precipitation step (e.g., as described in U.S. Pat. No. 10,190,075, which is incorporated by reference herein in its entirety).

Accordingly, in some preferred embodiments, the C16 fatty acid ethyl ester blends described above comprise less than 15.0%, 10.0%, 5.0%, 4.0%, 3.0%, 2.0% or 1.0% (or any range or value falling therein) C16:0 fatty acid ethyl esters on a w/w basis. In some particularly preferred embodiments, the C16 fatty acid ethyl ester blends comprise less than 5.0% C16:0 fatty acid ethyl esters. In some particularly preferred embodiments, the C16 fatty acid ethyl ester blends comprise less than 3.0% C16:0 fatty acid ethyl esters. In some particularly preferred embodiments, the C16 fatty acid ethyl ester blends comprise less than 2.0% C16:0 fatty acid ethyl esters.

In some preferred embodiments, the C18 fatty acid ethyl ester blends described above comprise less than 15.0%, 10.0%, 5.0%, 4.0%, 3.0%, 2.0% or 1.0% (or any range or value falling therein) C18:0 fatty acid ethyl esters on a w/w basis. In some particularly preferred embodiments, the C18 fatty acid ethyl ester blends comprise less than 5.0% C16:0 fatty acid ethyl esters. In some particularly preferred embodiments, the C18 fatty acid ethyl ester blends comprise less than 3.0% C18:0 fatty acid ethyl esters. In some particularly preferred embodiments, the C18 fatty acid ethyl ester blends comprise less than 2.0% C18:0 fatty acid ethyl esters.

In some preferred embodiments, the C20 fatty acid ethyl ester blends described above comprise less than 15.0%, 10.0%, 5.0%, 4.0%, 3.0%, 2.0% or 1.0% (or any range or value falling therein) C20:0 fatty acid ethyl esters on a w/w basis. In some particularly preferred embodiments, the C20 fatty acid ethyl ester blends comprise less than 5.0% C20:0 fatty acid ethyl esters. In some particularly preferred embodiments, the C20 fatty acid ethyl ester blends comprise less than 3.0% C20:0 fatty acid ethyl esters. In some particularly preferred embodiments, the C20 fatty acid ethyl ester blends comprise less than 2.0% C20:0 fatty acid ethyl esters.

In some preferred embodiments, the C22 fatty acid ethyl ester blends described above comprise less than 15.0%, 10.0%, 5.0%, 4.0%, 3.0%, 2.0% or 1.0% (or any range or value falling therein) C22:0 fatty acid ethyl esters on a w/w basis. In some particularly preferred embodiments, the C22 fatty acid ethyl ester blends comprise less than 5.0% C22:0 fatty acid ethyl esters. In some particularly preferred embodiments, the C22 fatty acid ethyl ester blends comprise less than 3.0% C22:0 fatty acid ethyl esters. In some particularly preferred embodiments, the C22 fatty acid ethyl ester blends comprise less than 2.0% C22:0 fatty acid ethyl esters.

In some preferred embodiments, the C24 fatty acid ethyl ester blends described above comprise less than 15.0%, 10.0%, 5.0%, 4.0%, 3.0%, 2.0% or 1.0% (or any range or value falling therein) C24:0 fatty acid ethyl esters on a w/w basis. In some particularly preferred embodiments, the C24 fatty acid ethyl ester blends comprise less than 5.0% C24:0 fatty acid ethyl esters. In some particularly preferred embodiments, the C24 fatty acid ethyl ester blends comprise less than 3.0% C24:0 fatty acid ethyl esters. In some particularly preferred embodiments, the C24 fatty acid ethyl ester blends comprise less than 2.0% C24:0 fatty acid ethyl esters.

In some preferred embodiments, the C26 fatty acid ethyl ester blends described above comprise less than 15.0%, 10.0%, 5.0%, 4.0%, 3.0%, 2.0% or 1.0% (or any range or value falling therein) C26:0 fatty acid ethyl esters on a w/w basis. In some particularly preferred embodiments, the C26 fatty acid ethyl ester blends comprise less than 5.0% C26:0 fatty acid ethyl esters. In some particularly preferred embodiments, the C26 fatty acid ethyl ester blends comprise less than 3.0% C26:0 fatty acid ethyl esters. In some particularly preferred embodiments, the C26 fatty acid ethyl ester blends comprise less than 2.0% C26:0 fatty acid ethyl esters.

The methods of the present invention are not limited to the use of ethyl ester blends prepared from any particular source. In some preferred embodiments, the ethyl esters are prepared from a vegetable oil. Suitable vegetable oils include, but are not limited to, safflower oil, sunflower oil, canola oil, corn oil, soybean oil, almond oil, coconut oil, cottonseed oil, flaxseed oil, grape seed oil, olive oil, palm oil, palm kernel oil, Cannabis oil, peanut oil, sesame oil and walnut oil. In some preferred embodiments, the ethyl esters are prepared from a marine oil. Suitable marine oil include, but are not limited to, algae oil, krill oil, fish oil, herring oil, herring roe oil, cod liver oil, squid oil, and Calanus oil.

In some further preferred embodiments, the present invention provides methods of fractionating lipids comprising a) dissolving the lipids in a solvent and b) spraying the dissolved lipids through a nozzle system to atomize the dissolved lipids and c) simultaneously flushing a stream of supercooled fluid air through the nozzle system to instantly cool the atomized blend and d) separating solid (precipitated) lipids from non-precipitated lipids.

These methods are not limited to the use of any particular solvent. In some preferred embodiments, the solvent is selected from or comprises any blend of water, methanol, ethanol, propanol, butanol, hexane, iso-octane, heptane, and acetone. Likewise, this method is not limited to the use of lipids from any particular source. In some preferred embodiments, the lipids are a vegetable oil. Suitable vegetable oils include, but are not limited to, safflower oil, sunflower oil, canola oil, corn oil, soybean oil, almond oil, coconut oil, cottonseed oil, flaxseed oil, grape seed oil, olive oil, palm oil, palm kernel oil, cannabis oil, peanut oil, sesame oil and walnut oil. In some preferred embodiments, the lipids are a marine oil. Suitable marine oil include, but are not limited to, algae oil, krill oil, fish oil, herring oil, herring roe oil, cod liver oil, squid oil, and Calanus oil.

In some further preferred embodiments, the present invention provides supplements, such as food supplements, or pharmaceutical comprising a product produced by the foregoing methods (e.g., a precipitated monounsaturated ethyl ester, a polyunsaturated fatty acid ethyl ester fraction, or a precipitated or non-precipitated lipid fraction).

In some embodiments, the products produced by the methods of this invention are contained in acceptable excipients and/or carriers for oral consumption. The actual form of the carrier, and thus, the composition itself, is not critical. The carrier may be a liquid, gel, gelcap, capsule, powder, solid tablet (coated or non-coated), tea, or the like. The composition is preferably in the form of a tablet or capsule and most preferably in the form of a soft gel capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, PA)

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: ascorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), Spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulphate or oxide; salt (sodium chloride); potassium chloride or acetate; ascorbic acid; ferric orthophosphate; niacinamide; zinc sulphate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulphate; vitamin A; vitamin C; inositol; potassium iodide. Suitable dosages for vitamins and minerals may be obtained, for example, by consulting the U.S. RDA guidelines.

In further embodiments, the compositions comprise at least one food flavoring such as acetaldehyde (ethanal), acetoin (acetyl methylcarbinol), anethole (parapropenyl anisole), benzaldehyde (benzoic aldehyde), N butyric acid (butanoic acid), d or 1 carvone (carvol), cinnamaldehyde (cinnamic aldehyde), citral (2,6 dimethyloctadien 2,6 al 8, gera nial, neral), decanal (N decylaldehyde, capraldehyde, capric aldehyde, caprinaldehyde, aldehyde C 10), ethyl acetate, ethyl butyrate, 3 methyl 3 phenyl glycidic acid ethyl ester (ethyl methyl phenyl glycidate, strawberry aldehyde, C 16 aldehyde), ethyl vanillin, geraniol (3,7 dimethyl 2,6 and 3,6 octadien 1 ol), geranyl acetate (geraniol acetate), limonene (d, l, and dl), linalool (linalol, 3,7 dimethyl 1,6 octadien 3 ol), linalyl acetate (bergamol), methyl anthranilate (methyl 2 aminobenzoate), piperonal (3,4 methylenedioxy benzaldehyde, heliotropin), vanillin, alfalfa (Medicago sativa L.), allspice (Pimenta officinalis), ambrette seed (Hibiscus abelmoschus), angelic (Angelica archangelica), Angostura (Galipea officinalis), anise (Pimpinella anisum), star anise (Illicium verum), balm (Melissa officinalis), basil (Ocimum basilicum), bay (Laurus nobilis), calendula (Calendula officinalis), (Anthemis nobilis), capsicum (Capsicum frutescens), caraway (Carum carvi), cardamom (Elettaria cardamomum), cassia, (Cinnamomum cassia), cayenne pepper (Capsicum frutescens), Celery seed (Apium graveolens), chervil (Anthriscus cerefolium), chives (Allium schoenoprasum), coriander (Coriandrum sativum), cumin (Cuminum cyminum), elder flowers (Sambucus canadensis), fennel (Foeniculum vulgare), fenugreek (Trigonella foenum graecum), ginger (Zingiber officinale), horehound (Marrubium vulgare), horseradish (Armoracia lapathifolia), hyssop (Hyssopus officinalis), lavender (Lavandula officinalis), mace (Myristica fragrans), marjoram (Majorana hortensis), mustard (Brassica nigra, Brassica juncea, Brassica hirta), nutmeg (Myristica fragrans), paprika (Capsicum annuum), black pepper (Piper nigrum), peppermint (Mentha piperita), poppy seed (Papayer somniferum), rosemary (Rosmarinus officinalis), saffron (Crocus sativus), sage (Salvia officinalis), savory (Satureia hortensis, Satureia montana), sesame (Sesamum indicum), spearmint (Mentha spicata), tarragon (Artemisia dracunculus), thyme (Thymus vulgaris, Thymus serpyllum), turmeric (Curcuma longa), vanilla (Vanilla planifolia), zedoary (Curcuma zedoaria), sucrose, glucose, saccharin, sorbitol, mannitol, aspartame. Other suitable flavoring are disclosed in such references as Remington's Pharmaceutical Sciences, 18th Edition, Mack Publishing, p. 1288-1300 (1990), and Furia and Pellanca, Fenaroli's Handbook of Flavor Ingredients, The Chemical Rubber Company, Cleveland, Ohio, (1971), known to those skilled in the art.

In other embodiments, the compositions comprise at least one synthetic or natural food coloring (e.g., annatto extract, astaxanthin, beet powder, ultramarine blue, canthaxanthin, caramel, carotenal, beta carotene, carmine, toasted cottonseed flour, ferrous gluconate, ferrous lactate, grape color extract, grape skin extract, iron oxide, fruit juice, vegetable juice, dried algae meal, tagetes meal, carrot oil, corn endosperm oil, paprika, paprika oleoresin, riboflavin, saffron, tumeric, tumeric and oleoresin).

In still further embodiments, the compositions comprise at least one phytonutrient (e.g., soy isoflavonoids, oligomeric proanthcyanidins, indol 3 carbinol, sulforaphone, fibrous ligands, plant phytosterols, ferulic acid, anthocyanocides, triterpenes, omega 3/6 fatty acids, conjugated fatty acids such as conjugated linoleic acid and conjugated linolenic acid, polyacetylene, quinones, terpenes, cathechins, gallates, and quercitin). Sources of plant phytonutrients include, but are not limited to, soy lecithin, soy isoflavones, brown rice germ, royal jelly, bee propolis, acerola berry juice powder, Japanese green tea, grape seed extract, grape skin extract, carrot juice, bilberry, flaxseed meal, bee pollen, Ginkgo biloba, primrose (evening primrose oil), red clover, burdock root, dandelion, parsley, rose hips, milk thistle, ginger, Siberian Ginseng, rosemary, curcumin, garlic, lycopene, grapefruit seed extract, spinach, and broccoli.

In still other embodiments, the compositions comprise at least one vitamin (e.g., vitamin A, thiamin (B1), riboflavin (B2), pyridoxine (B6), cyanocobalamin (B12), biotin, ascorbic acid (vitamin C), retinoic acid (vitamin D), vitamin E, folic acid and other folates, vitamin K, niacin, and pantothenic acid). In some embodiments, the particles comprise at least one mineral (e.g., sodium, potassium, magnesium, calcium, phosphorus, chlorine, iron, zinc, manganese, fluorine, copper, molybdenum, chromium, selenium, and iodine). In some particularly preferred embodiments, a dosage of a plurality of particles includes vitamins or minerals in the range of the recommended daily allowance (RDA) as specified by the United States Department of Agriculture. In still other embodiments, the particles comprise an amino acid supplement formula in which at least one amino acid is included (e.g., l-carnitine or tryptophan).

### EXPERIMENTAL

### Example 1

A distilled C16 fraction of fatty acid ethyl esters obtained from a fish oil was injected into precooled ethanol. The fatty acid ester blend was at room temperature whereas the ethanol temperature was about −80° C. The blend was shaken a few times before it was filtrated at −35° C. to remove palmitic acid. The filtrate was further cooled to −70° C. and again filtrated. The solid crystals were recovered, and solvent was removed in a rotavapor at 150 mbar and 50° C. The obtained lipid fraction comprised about 90% palmitoleic acid.

### Example 2

A blend of fatty acid ethyl esters comprising 52% saturated and monounsaturated C16 fatty acids along with 48% polyunsaturated C16 fatty acid ethyl esters in ethanol, was sprayed through a nozzle with a dual inlet system such that both the stream of said blend and a stream of fluid nitrogen was flushed through the nozzle simultaneously. A dust of precipitated lipids along with instantly cooled ethanol was filled into the insulated receiving tank. The instantly cooled blend was allowed to rest for 30 minutes before the tank was emptied through a filter applying a vacuum on the filtrate receiving tank. The fatty acid esters collected in the receiving tank, comprised above 90% polyunsaturated 16 carbon fatty acids.

### Example 3

An ethyl ester fraction containing unsaturated C 16 fatty acid ethyl esters was diluted 1:10 (w/w) in ethanol and cooled to −70 C. Precipitated material was removed in a filter. The blend before and the precipitate was analysed by GC-FID to provide the composition (area %) of ethyl esters tabulated below. The precipitate contained 78.8% palmitoleic acid (C16:1 n-7).

| fatty acid ethyl ester | before | precipitate |
|---|---|---|
| C 16:0 | 2.2 | 2.6 |
| C16:1 n-7 | 52.8 | 78.8 |
| C16:2 | 8.8 | 2.6 |
| C16:3 | 9.4 | 1.7 |
| C16:4 | 13.8 | 2.4 |

### LITERATURE

1) U.S. Pat. No. 10,190,075 B2 Byelashov et. al., Enrichment of palmitoleic acid and Palmitoleic acid derivatives by dry and solvent-aided winterization.
2) Xiniy Cheng et. al. (2021) Enrichment of palmitoleic acid by a combination of two step solvent crystallization and molecular distillation. J. Oleo Sci. 70, (5) 599-606.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

References, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A method of separating a fraction containing a monounsaturated fatty acid ethyl ester from a fraction containing polyunsaturated fatty acid ethyl esters of equal carbon-number in a fatty acid ethyl ester blend containing the monounsaturated and polyunsaturated fatty acid ethyl esters of equal carbon-number comprising:
1) cooling a mixture of the blend in ethanol to a temperature at which the monounsaturated fatty acid precipitates to provide a precipitated fraction comprising the monounsaturated fatty acid and a fraction comprising the polyunsaturated fatty acid ethyl esters,
2) separating the precipitated fraction from the non-separated fraction, and
3) removing the ethanol solvent by vacuum distillation to provide the fraction comprising polyunsaturated fatty acid ethyl esters.

2. The method of claim 1, wherein the fatty acid ethyl ester blend and ethanol are combined in a ratio of between 1:3 (w/w) and 1:20 (w/w).

3. The method of claim 1, wherein the fatty acid ethyl ester blend comprises fatty acid moieties having a length of 16 carbons and the fraction comprising polyunsaturated fatty acid ethyl esters comprises a blend of C16:2, C16:3 and/or C16:4 ethyl esters.

4. The method of claim 1, wherein the precipitated monounsaturated fatty acid ethyl ester is a C16:1 ethyl ester.

5. The method of claim 1, wherein the fatty acid ethyl ester blend comprises fatty acid moieties having a length of 20 carbons and fraction comprising polyunsaturated fatty acid ethyl esters comprises a blend of C20:2, C20:3, C20:4 and/or C20:5 ethyl esters.

6. The method of claim 5, wherein the precipitated fatty acid ethyl ester is a C20:1 ethyl ester.

7. The method of claim 1, wherein the fatty acid ethyl ester blend comprises fatty acid moieties having a length of 22 carbons fraction comprising polyunsaturated fatty acid ethyl esters comprises a blend of C22:2, C22:3, C22:4 and/or C22:5 ethyl esters.

8. The method of claim 7, wherein the precipitated monounsaturated fatty acid ethyl ester is a C22:1 ethyl ester.

9. The method according to claim 1, wherein the fatty acid ethyl ester blend containing the monounsaturated and polyunsaturated fatty acid ethyl esters has been treated by a previous step where unsaturated fatty acid ethyl esters are removed or reduced in concentration.

10. The method according to claim 1, wherein the fatty acid ester blend is injected into the ethanol.

11. The method of claim 10, wherein the injection of the fatty acid ethyl ester into the ethanol is performed through a nozzle or microtubular device.

12. The method according to claim 10, wherein the ethanol is cooled prior to injection of the fatty acid ethyl ester blend.

13. The method according to claim 12, wherein the precooled ethanol is cooled to between minus 50° C. and minus 90° C. before injecting the fatty acid ethyl ester blend.

14. The method according to claim 11, wherein the fatty acid ethyl ester blend is sprayed through a nozzle that is cooled by simultaneously flushing fluid nitrogen through the nozzle.

15. The method according to claim 12, wherein the fatty acid ethyl ester blend that is injected into the cooled ethanol solution comprises a premixture of fatty acid ethyl esters and ethanol in a ratio of about 1:1 (w/w).

16. The method according to claim 1, wherein the fatty acid ethyl esters are derived from a vegetable oil or a marine oil.

17. The method according to claim 16, wherein the marine oil is derived from a fish, algae, or krill.

18. A method of fractionating lipids comprising a) dissolving the lipids in a solvent and b) spraying the dissolved lipids through a nozzle system to atomize the dissolved lipids and c) simultaneously flushing a stream of supercooled fluid air through the nozzle system to instantly cool the atomized blend and d) separating solid (precipitated) lipids from non-precipitated lipids.

19. The method of claim 18, wherein the air comprises more than 95% nitrogen.

20. The method according to claim 18, wherein the solvent is selected from or comprise any blend of water, methanol, ethanol, propanol, butanol, hexane, iso-octane, heptane, and acetone.

21-22. (canceled)

23. A food supplement product comprising a product obtained by the method of claim 1.

24. A pharmaceutical product comprising a product obtained by the method of claim 1.
